(19) United States
(12) Patent Application Publication
(10) Pub. No.: US 2018/0064400 A1
(43) Pub. Date: Mar. 8, 2018

(54) CARDIOVASCULAR DETERIORATION WARNING SCORE

(71) Applicant: KONINKLIJKE PHILIPS N.V., EINDHOVEN (NL)

(72) Inventors: Nicolas Wadih CHBAT, WHITE PLAINS, NY (US); Ronaldus Maria AARTS, GELDROP (NL); Sophia Huai ZHOU, CAMBRIDGE, MA (US)

(21) Appl. No.: 15/559,608

(22) PCT Filed: Apr. 8, 2016
(86) PCT No.: PCT/IB2016/051997

§ 371 Date: Sep. 19, 2017

Related U.S. Application Data
(60) Provisional application No. 62/144,372, filed on Apr. 8, 2015.

(51) Int. Cl. A61B 5/00; A61B 5/0205; A61B 5/0452; A61B 5/1455; G06F 19/00

(52) U.S. Cl. CPC A61B 5/7275; A61B 5/0205; A61B 5/0452; A61B 5/091; A61B 5/7267; G06F 19/3431; A61B 5/14551

(57) ABSTRACT

A patient monitor (12) includes a display (14) and sensors (20, 22, 24) reading vital signs of a human subject. In a cardiovascular early warning scoring (cEWS) method, the human subject is classified using a plurality of cardiovascular deterioration classifiers (52, 152, 54, 56, 58) each trained respective to a different type of cardiovascular deterioration. The cardiovascular deterioration classifiers operate on a set of inputs characterizing the human subject including the at least one cardiovascular parameter (42) and the at least one respiratory parameter (44), such as tidal volume read by an airflow sensor (24). The cardiovascular early warning scores for the different types of cardiovascular deterioration are outputted on the display of the patient monitor. An empirical myocardial ischemia classifier (52) may be combined with at least one additional ischemia score generated by applying a set of rules (160) or a physiological model (162).

### FIELD

The following relates generally to the cardiac care arts, medical emergency response arts, and so forth.

### BACKGROUND

Numerous clinical scenarios may arise which might, or might not, be indicative of cardiac deterioration, such as a patient having one or more of the symptoms: feeling dizzy; physical weakness; rapid or irregular heartbeats; shortness of breath; discomfort in the chest; or so forth. Such symptoms are common cardiovascular disease symptoms of patients with potential risks for cardiac arrest, or acute heart attack, or acute heart failure when the symptoms are severe, or for irregular heart rhythm, or coronary artery disease when the symptoms are less severe. Typical situations in which cardiac deterioration is more likely to be present include patients being transported by an ambulance, or admitted to an emergency department of a hospital, or a hospitalized patient after a knee replacement surgery or other surgical or other stressful medical procedure.

Early detection and diagnosis of cardiac deterioration has a significant impact on the ultimate success or failure of cardiac care. Cardiac deterioration mechanisms directly associated with the cardiac muscle include, for example: myocardial ischemia (a reduction in blood flow/oxygenation of the heart), left ventricular hypertrophy (muscle buildup in the left ventricular wall, usually resulting from chronic excessive cardiac effort due to high blood pressure or another condition), systolic heart failure (deterioration in ventricular performance during systolic pumping action, usually correlating with low ejection fraction), and diastolic heart failure (deterioration in ventricular performance during diastole relaxation, usually correlating with low stroke volume). Other cardiac deterioration mechanisms relate to the vasculature servicing the heart, such as valve degradation, or plaque build-up which can lead to stenosis. The appropriate treatment depends upon which of these various cardiac deterioration mechanisms (or combination of mechanisms) is present. Many of these cardiac deterioration mechanisms, if left untreated, can lead to acute debilitating or life-threatening medical events such as cardiac arrest, acute heart attack, acute heart failure, irregular heart rhythm, coronary artery disease, or the like.

Numerous specialized medical tests have been developed to diagnose cardiac deterioration. In practice, however, these are often not ordered for a given patient until the cardiac deterioration has reached an advanced state and has become manifestly symptomatic. Moreover, interpretation of various cardiac tests is difficult, and in the early stages of cardiac deterioration the physician seeing the patient is often not a trained cardiologist but rather a general practice (GP) physician and/or a physician specializing in some other area.

The following discloses a new and improved systems and methods that address the above referenced issues, and others.

### SUMMARY

In one disclosed aspect, a patient monitor is disclosed, comprising a display component, a plurality of sensors reading vital signs of a human subject including at one cardiovascular parameter and at least one respiratory parameter, and a microprocessor or microcontroller programmed to perform a cardiovascular early warning scoring (cEWS) method. The cEWS method includes the operations of: (i) classifying the human subject using a plurality of cardiovascular deterioration classifiers each trained to classify the human subject respective to a different type of cardiovascular deterioration to generate cardiovascular early warning scores for the different types of cardiovascular deterioration, the plurality of cardiovascular deterioration classifiers operating on a set of inputs characterizing the human subject including the at least one cardiovascular parameter and the at least one respiratory parameter read by the plurality of sensors; and (ii) outputting the cardiovascular early warning scores for the different types of cardiovascular deterioration on the display component of the patient monitor. The set of inputs may include the at least one cardiovascular parameter read by electrocardiograph electrodes and the at least one respiratory parameter comprising tidal volume read by an airflow sensor.

In another disclosed aspect, a non-transitory storage medium stores instructions readable and executable by a patient monitor comprising a plurality of sensors, a display component, and a microprocessor or microcontroller to perform a myocardial ischemia early warning method as follows. Vital sign data for a human subject are acquired using the plurality of sensors. The human subject is classified to generate an empirical myocardial ischemia score using an empirical myocardial ischemia classifier trained on a labeled data set representing training subjects with each training subject i represented by a vector x̅ᵢ of features of the training subject i and a label yᵢ representing a state of myocardial ischemia in the training subject i. The classifying includes inputting a vector to the empirical myocardial ischemia classifier that includes features generated from the acquired vital sign data for the human subject. At least one additional myocardial ischemia score is generated by applying a set of rules or a physiological model to a set of inputs characterizing the human subject including inputs generated from the acquired vital sign data for the human subject. A combined myocardial ischemia score is generated comprising a weighted combination of the empirical myocardial ischemia score and the at least one additional myocardial ischemia score. A representation of the combined myocardial ischemia score is displayed on the display component of the patient monitor.

One advantage resides in facilitating early diagnosis of cardiovascular deterioration, and facilitating early identification of the type of cardiovascular deterioration.

Another advantage resides in providing early diagnosis of myocardial ischemia.

Another advantage resides in providing the foregoing while leveraging and providing the context of a rules-based diagnosis that is heuristic in nature.

Another advantage resides in synergistically combining multiple automated pathways to provide more accurate diagnosis of cardiovascular deterioration.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 diagrammatically illustrates a medical monitoring system including a cardiovascular deterioration early warning system.

FIG. 2 shows resting heart rate tables for men and women.

FIG. 3 plots maximum, average, and minimum values for systolic and diastolic blood pressure as a function of age.

FIG. 4 diagrammatically illustrates a combinational variant of the cardiovascular deterioration early warning system of FIG. 1, for the illustrative example of providing early warning of myocardial ischemia.

### DETAILED DESCRIPTION

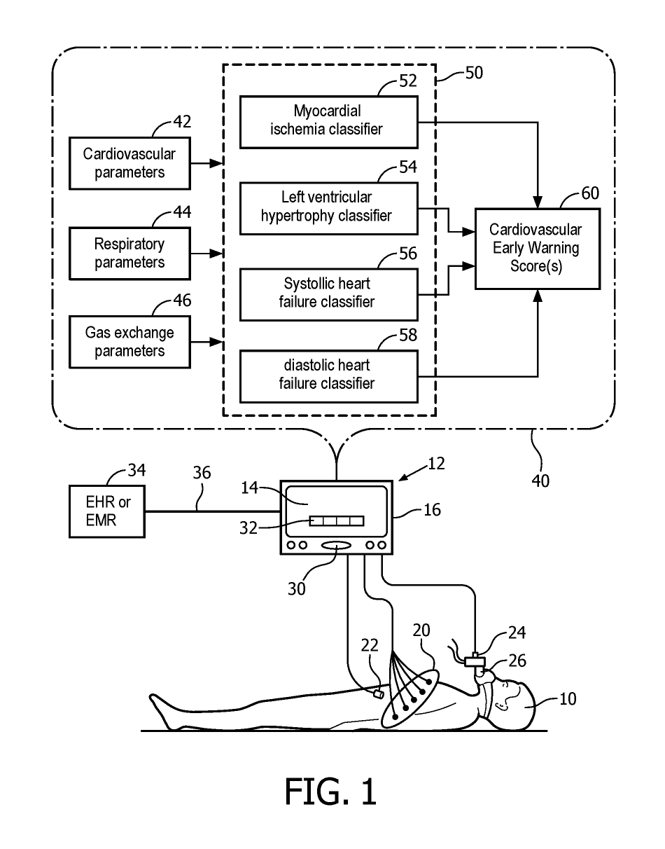

With reference to FIG. 1, a human subject 10 (such as an in-patient admitted to a hospital, or a nursing home resident, or an outpatient or so forth) is monitored by a patient monitor 12 comprising a display device or component 14, a microprocessor or microcontroller (not shown, but typically housed in a monitor housing 16), and a plurality of physiological (i.e. vital sign) sensors 20, 22, 24 such as an illustrative set of electrocardiograph electrodes 20, a pulse oximeter sensor 22 (not connected as illustrated but typically clipped onto a finger or earlobe of the subject 10), and an airflow sensor 24. In illustrative FIG. 1 the airflow sensor 24 is installed in a full-face mask 26 used for mechanical ventilation, sleep apnea treatment, general respiratory monitoring or the like. In other embodiments the airflow sensor 24 may be installed in conjunction with a nasal mask or nasal cannula or the like. In general, the patient monitor 12 may be in one of a number of typical settings, such as being installed in a patient room at a hospital (if the subject 10 is an in-patient, could be a specialized hospital area such as the surgical floor, post-anesthesia care unit, intensive care unit, or so forth), or at a nursing home room (if the subject 10 is a nursing home resident), in an ambulance or other emergency response system (EMS) vehicle (if the subject 10 is the subject of an EMS call), or so forth,

The sensors 20, 22, 24 acquire vital sign data in real-time (i.e. continuously, or by sampling with a relatively fast sampling rate) with the vital sign data being optionally processed by algorithms running on the microprocessor or microcontroller of the patient monitor 12. For example, the patient monitor 12 may execute algorithms to generate ECG lead traces from measured voltages of the ECG electrodes 20 and to extract information from the ECG lead traces such as heart rate and presence/absence of associated rate abnormalities (e.g. tachycardia, bradycardia, may use age- and/or gender-specific limits), heart rate variability, QT interval, presence/absence of various arrhythmias such as atrial (AFib), supraventricular tachycardia (SVT), increased QT interval (long QTc), or so forth. The patient monitor 12 may execute algorithms to process the airflow data acquired by the airflow sensor 24 to extract information such as respiratory rate and tidal volume. As another example, the patient monitor 12 may execute algorithms to process a peripheral plethysmograph waveform acquired by the pulse oximeter to derive saturation of peripheral oxygen (SpO₂) and heart rate data.

The vital sign sensors 20, 22, 24 are merely illustrative, and additional or other vital sign sensors are contemplated, such as a blood pressure cuff, sphygmomanometer, or other blood pressure sensor or sensors. The patient monitor 12 may process blood pressure date to extract systolic and diastolic blood pressure, with high- or low-blood pressure limits defined that may again be age- and/or gender-specific. Furthermore, if the illustrative mask 26 is part of a mechanical ventilation system (that is, the patient 10 is mechanically ventilated) then other ventilator data additional to the previously mentioned respiration rate and tidal volume may be available and suitably input to the patient monitor 12.

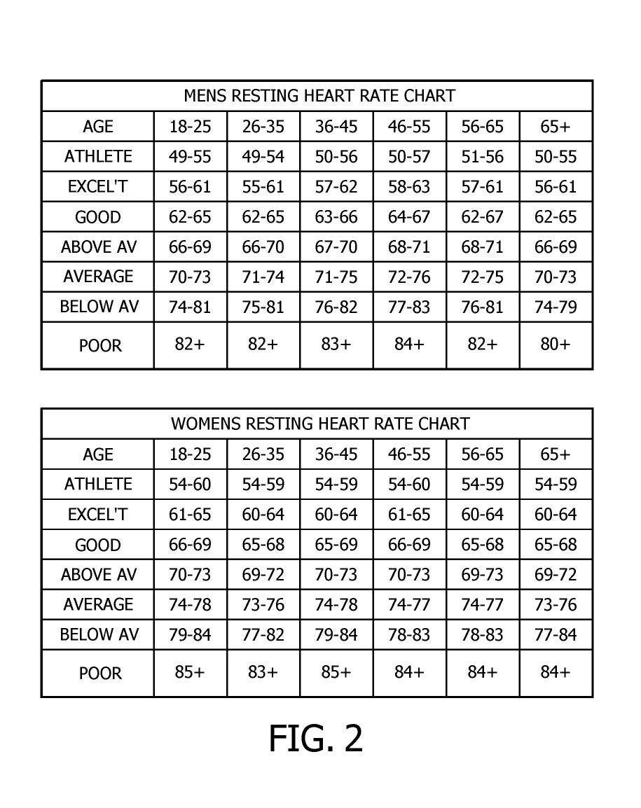

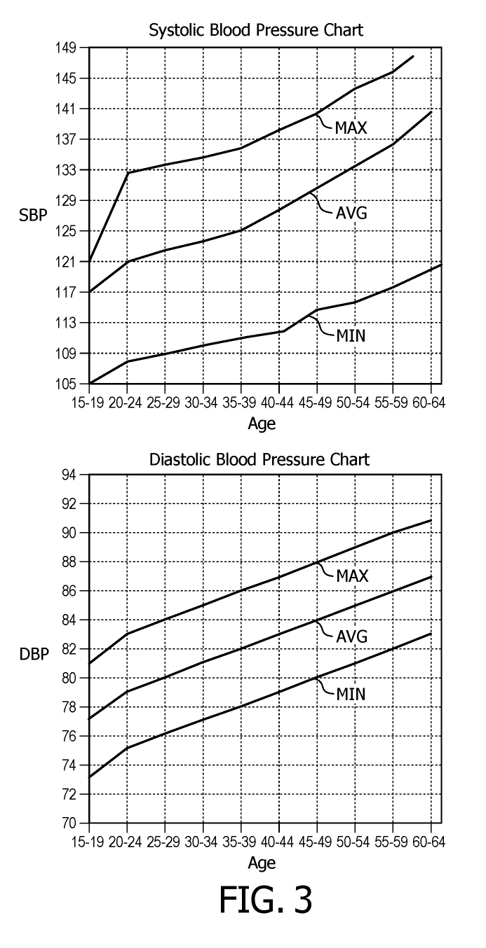

With brief reference to FIGS. 2 and 3, the impact of demographic data on vital signs is illustrated. FIG. 2 shows resting heart rate tables for men and women, further categorized by age and fitness level. FIG. 3 plots systolic and diastolic blood pressure (maximum, average, and minimum values) as a function of age.

With reference back to FIG. 1, the patient monitor 12 is also capable of receiving and storing patient physiological data acquired by laboratory tests or the like. For example, blood gas analysis test results may be received, providing information such as partial pressure of oxygen (PaO₂) and/or partial pressure of carbon dioxide (PaCO₂). Another contemplated laboratory result is troponin level in the blood. Such data may be entered into the patient monitor 12 manually, e.g. using a physical keypad 30 or soft keys 32 on the display 14 (the soft keys 32 are implementable if the display 14 is a touch-sensitive display). In one embodiment, respiratory rate is determined manually, for example by a nurse visually assessing respiratory rate, and the manually determined respiratory rate is entered into the patient monitor 12 using the input(s) 30, 32. Other patient data such as demographic data (e.g. age, gender), medical history, perioperative status data, and the like may be similarly provided to the patient monitor 12. Rather than being manually entered via a user interface 30, 32 of the patient monitor 12, such information may be input to the patient monitor 12 from an Electronic Health Record (EHR), Electronic Medical Record (EMR), or the like 34 via a hospital data network or other electronic data network 36 if the patient monitor 12 is connected with such patient records storage and communication infrastructure 34, 36.

The illustrative patient monitor 12 further includes a cardiovascular deterioration early warning scoring (cEWS) (sub-)system 40 that is diagrammatically depicted in FIG. 1 by functional blocks suitably executed by the microprocessor or microcontroller of the patient monitor 12. The illustrative cEWS system 40 receives as inputs patient values (i.e. vital sign readings or sensor values, possibly processed) for cardiovascular parameters 42 such as ECG-derived data, heart rate from the pulse oximeter 22), blood pressure data, or so forth. Such parameters (or at least a sub-set of them) are readily recognized as being pertinent to assessing cardiovascular deterioration.

Additionally, the cEWS system 40 receives at least one respiratory parameter 44, such as respiratory rate, tidal volume, or so forth. The illustrative cEWS system 40 also receives at least one gas exchange parameter 46, such as SpO₂ from the pulse oximeter 22, or PaO₂ and/or PaCO₂ values from blood gas analysis, or so forth. It is recognized herein that these additional parameters 44, 46, although not characterizing the cardiovascular system directly, are of value in assessing cardiovascular deterioration because the respiratory and gas exchange systems characterize the pulmonary system which together with the cardiovascular system forms an integrated cardiopulmonary system.

The cEWS system 40 comprises a plurality of cardiovascular deterioration classifiers (i.e. inference engines) 50, one for each type of cardiovascular deterioration of interest. The illustrative cEWS system 40 includes a myocardial ischemia classifier 52, a left ventricular hypertrophy classifier 54, a systolic heart failure classifier 56, and a diastolic heart failure classifier 58. There are merely illustrative, and classifiers trained to detect other types of cardiovascular deterioration such as cardiac valve deterioration, low cardiac output, cardiac arterial stenosis, or so forth are additionally or alternatively contemplated. Each classifier 52, 54, 56, 58 may, for example, be a neural network, support vector machine, a nonlinear regression model (e.g. logistic or polynomial regression), or other type of classifier. It will be appreciated that the classifiers 52, 54, 56, 58 may in general be of different types.

In general, each classifier 52, 54, 56, 58 is trained using a labeled data set {(x̅ᵢ, yᵢ)}, i=1, . . . , N comprising N past (training) patients, with each training patient i being represented by a vector x̅ᵢ of patient data (e.g. vital signs, demographic data, patient history data) and a label yᵢ. The elements of the training patient data vector x̅ᵢ are referred to herein as “features” in accord with common usage in the classifier training arts. The label yᵢ represents whether the training patient i was diagnosed with the cardiac deterioration for which the classifier is being trained. For example, in training the ischemia classifier 52 the label yᵢ may be a binary value indicating whether the training patient i was diagnosed with cardiac ischemia. Alternatively, the label may be more informative, e.g. the label yᵢ for training the ischemia classifier 52 may be integer value in the range between 0 and 5, where a value of 0 indicates the training patient was diagnosed with no detectable cardiac ischemia, a value of 5 indicates the training patient was diagnosed with ischemia of highest severity, and the values 1, . . . , 4 denote ischemia in the training patient at intermediate severity levels. Continuous outputs are also contemplated, e.g. in a range [0,1] where 0 indicates no detectable ischemia and 1 indicates highest severity ischemia. The classifier is trained to minimize an error metric between its outputs (i.e., “predictions” ŷᵢ) for input training patient data sets x̅ᵢ and the corresponding actual (a priori known) labels yᵢ. For example, a simple least squared error of the form Σᵢ₌₁^{N}(ŷᵢ−yᵢ)² may be used. The trained classifier outputs predictions ŷ in a format (binary, multi-level, or so forth) which may in general be different for each of the different classifiers 52, 54, 56, 58.

The trained classifiers 52, 54, 56, 58 are applied to the patient 10, who is not one of the training patients, and for whom the status of cardiac deterioration (if any) is not known a priori. In applying the classifiers 52, 54, 56, 58 to the patient 10, the patient data 42, 44, 46 are formulated in the same manner as the training patient data vectors x̅ᵢ. It should be noted that the format and/or content of the vector x̅ᵢ may be different for each different classifier 52, 54, 56, 58. For example, some training approaches employ a features reduction operation, or some features that are not expected to be relevant to the mode of cardiac deterioration under training may be omitted, so that the vector x̅ᵢ for that classifier is some sub-set of the available patient data 42, 44, 46. The output of each type-specific classifier 52, 54, 56, 58 is a prediction ŷ of whether the subject 10 has that type of cardiac deterioration, or if the output is multi-level or continuous the prediction ŷ encompasses the level of severity of that type of cardiac deterioration in the subject 10. It should be noted that the predictions ŷ may have a different format from the labels yᵢ—for example, the labels may be binary values (0=patient not diagnosed with this type of cardiac deterioration; 1=patient was so diagnosed) but the predictions ŷ may be continuous values in the range [0,1]. A continuous-valued prediction in the range [0,1] advantageously may be interpreted as a probability and, for example, written as a percentage in the range 0-100%.

In some embodiments, the prediction outputs may be tied to clinical guidelines used by the ER, EMS, or other medical provider. For example, in an EMS call setting, if the myocardial ischemia score is sufficiently high the output may (in addition to identifying a probable ischemia condition) present the ischemia therapy called for in the clinical guideline for treating ischemia.

It is contemplated that the classifiers 52, 54, 56, 58 may be re-trained occasionally to more accurately reflect current patient demographics.

The use in the cEWS system 40 of the plurality of classifiers 52, 54, 56, 58, one for each type of cardiac deterioration of interest, recognizes that different types of cardiac deterioration, though somewhat interrelated, have distinct characteristics, so that a single classifier would be unlikely to be effective. The output predictions ŷ of the set of classifiers 52, 54, 56, 58 may be variously combined and/or presented as one or more cardiovascular early warning scores 60. In one approach, only the highest (i.e. most severe) prediction (score) is presented, and then only if that highest prediction is higher than some threshold. This approach is particularly advantageous in a setting such as an emergency room (ER) or emergency medical service (EMS) call, where medical personnel are dealing with a triage situation and need to be made aware of only the most severe condition. In a variant approach also suitable for triage situations, each classifier score is presented individually but only if its value (i.e. severity) is greater than some (possibly type-specific) threshold. To reduce the information that needs to be processed by emergency medical personnel, it is further contemplated to present the predictions (scores) in some discretized fashion, for example a value of “HIGH” or “MODERATE” depending on the score. Other readily perceived formats are contemplated, such as displaying each prediction as a slider or scale running (for example), with the low end labeled to indicate no likelihood of that type of cardiac deterioration and the high end labeled to indicate a high likelihood of that type of cardiac deterioration. Color coding may also be used, e.g. displaying high scores in red, moderate scores in yellow, and low scores in green. The scores are suitably displayed on the display 14 of the patient monitor 12, although other outputs are contemplated such as an audible alarm in the case of a very high score. In embodiments suited for non-emergency situations, it is contemplated to present all cEWS values, e.g. as percent probabilities or other numerical values. More generally, the cEWS values can be used for continuous monitoring, for example displayed as a trend line, numeric values updated in real time, or so forth in an ambulance or other mobile emergency response setting, at the hospital room bedside, at a nurses' station, or so forth.

The cEWS system 40 diagrammatically shown in FIG. 1 is a data-driven system that relies upon empirically trained inference engines 52, 54, 56, 58 to provide predictions (i.e. early warning scores) of various types of cardiac deterioration. The approach provides scores for different types of cardiac deterioration, thus enabling medical personnel without cardiac specialization to make an early assessment of incipient cardiac degradation so that a more detailed cardiac assessment can be performed. It should be noted that the cardiac deterioration scores are not medical diagnoses, but rather early warning indicators which may be considered by a physician, along with other information such as a physical examination, various laboratory test results, and so forth to guide initial triage and/or assist the physician in early detection of cardiovascular deterioration. In general, it is expected that more detailed cardiac assessment will be triggered by the early warning provided by the cEWS system 40 in order to obtain a diagnosis of any cardiac deterioration actually present in the patient 10.

One possible difficulty with the cEWS system 40 of FIG. 1 is that, as a purely empirical system, its operation is not as transparent as, for example, heuristic diagnostic rules commonly relied upon by physicians. The empirical approach may also be difficult to correlate with the underlying physiology. This can introduce certain difficulties. The lack of readily apparent correlation with heuristic diagnostic rules and underlying physiology may cause medical personnel to resist relying upon the early warning scores generated by the cEWS system 40. Also, there may be disadvantages to substituting the cEWS system 40 for existing heuristic diagnostic rules or first-principles physiological analysis. For example, inference engines can suffer from excessive random error if the training data set is too small, or can suffer from systematic error if there are systematic deficiencies in the training data, such as a systematic predisposition to over-diagnose (or under-diagnose) a particular type of cardiac deterioration which is captured in the annotated labels yᵢ for that type. The empirical training can also capture spurious correlations.

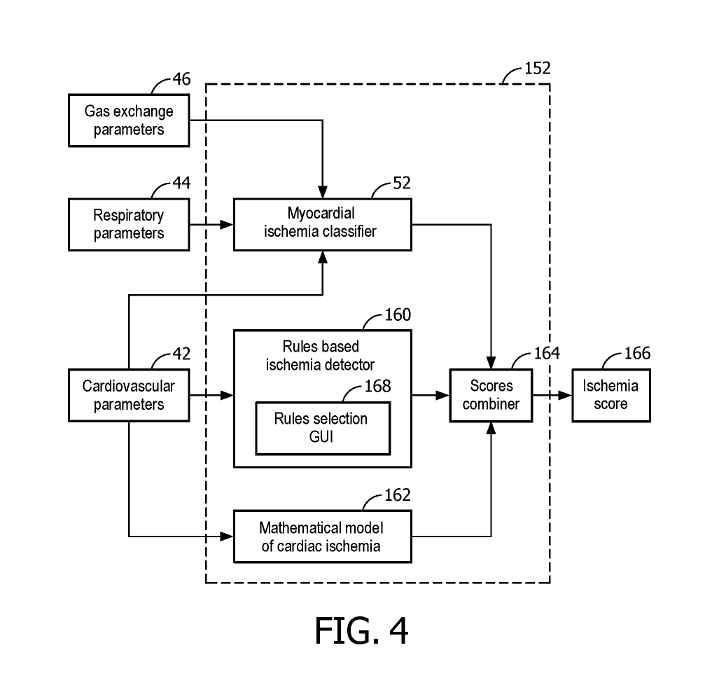

With reference to FIG. 4, these difficulties are addressed in a variant embodiment of the cardiovascular deterioration type-specific classifiers 52, 54, 56, 58. In illustrative FIG. 4, a variant embodiment of 152 the myocardial ischemia classifier 52 of FIG. 1 is described; however, the extensions to the myocardial ischemia classifier 52 described herein with reference to FIG. 4 are readily applied to any of the other cardiovascular deterioration type-specific classifiers 54, 56, 58. As seen in FIG. 4, the variant myocardial ischemia classifier 152 incorporates the myocardial ischemia classifier 52 as a component, and this component (as in FIG. 1) receives as inputs the cardiovascular parameters 42, at least one respiratory parameter 44, and optionally at least one gas exchange parameter 46. The cardiac ischemia classifier 152 further incorporates two additional cardiac ischemia detection components: a codified rules-based cardiac ischemia detector 160 and a physiological model component that mathematically models the physiology and progression of cardiac ischemia 162. A scores combiner 164 combines the outputs of the constituent myocardial ischemia detectors 52, 160, 162, for example using a weighted sum of their outputs, to generate an ischemia score 166 that may be output by itself as a myocardial ischemia detector, or may be employed in the cEWS system 40 of FIG. 1 in place of the output of the empirical myocardial ischemia classifier 52. (In other words, the modified ischemia detector 152 of FIG. 4 may be substituted for the ischemia classifier 52 of FIG. 1).

In the approach of FIG. 4, the rules-based ischemia detector 160 provides the physician with a familiar component. Various heuristic rules are employed by different cardiologists, different hospitals, or so forth. The rules used by a particular cardiologist may be a standard set of rules, such as the rules promulgated in the 2013 ACC/AHA guideline on the assessment of cardiovascular risk: a report of the American College of Cardiology/American Heart Association Task Force on Practice Guidelines. http://circ.ahajournals.org/content/early/2013/11/11/01.cir.0000437741.48606.98). On the other hand, a given cardiologist or hospital may employ a variant of the standard rules, or may prefer to employ a standard set of rules promulgated by a different authority. To accommodate such individual and/or institutional preferences, as we as to reassure the cardiologist regarding which rules are being employed, the illustrative rules-based ischemia detector 160 includes a rules selection graphical user interface (GUI) 168 via which the user can select from amongst one, two, three, or more standard rules sets (e.g. the 2013 ACC/AHA 2013 guideline, an earlier edition of the ACC/AHA guideline, and/or a guideline promulgated by another cardiovascular care authority). The GUI 168 is suitably implemented by the microprocessor or microcontroller patient monitor 12 programmed to implement the GUI 168 using the display device or component 14 and the user input device(s) 30, 32.

In one illustrative implementation, of the myocardial ischemia detector of FIG. 4, the set of inputs 42, 44, 46 includes troponin levels, blood pressure, disease history, sex, age, and other demographic information like Body Mass Index (BMI) and others and so forth. Optionally, an automated feature selection algorithm employing a regression-type model is employed to identify the most probative features for detecting ischemia, which compares its fitting results with training data. A fit metric such as the correlation coefficient or the coefficient of variation can be used to judge the quality of the fit. The features included in the best fit model will then serve as the features or guides to the three ischemia detector components 52, 160, 162 which are described in turn below.

The ischemia classifier 52, already described with reference to FIG. 1, is a data-driven component. The data-based algorithm can, for example, be a data mining, machine learning, or statistical correlation type model for ischemia detection. Examples of such algorithms include neural network or logistic regression classifiers.

The rules-based ischemia detector 160 is a codification of the heuristic rules used by the physician in performing ischemia detection. The rules can be codified using a fuzzy inference engine where heuristic rules are translated into mathematical formulation giving crisp features to be selected. Some suitable rules that could be implemented via the rules-based ischemia detector 160 include the aforementioned 2013 ACC/AHA guideline, and/or the AHA/ACCF/HRS Recommendations for the Standardization and Interpretation of the Electrocardiogram Part VI: Acute Ischemia/Infarction (Circulation. 2009; 119:e262-e27). In typical guidelines for cardiovascular deterioration, the guidelines rely upon cardiovascular parameters, but typically not on respiratory or gas exchange parameters. Accordingly, the illustrative rules-based ischemia detector 160 receives as input the cardiovascular parameters 42 but not the at least one respiratory parameter 44 and not the at least one gas exchange parameter 46. (However, it is also contemplated for the rules-based ischemia detector to employ rules additionally operating on respiratory and/or gas exchange parameter(s)).

The physiological model component 162 comprises static (algebraic) and/or dynamic (differential) equations that articulate ischemic deterioration of the myocardium. This knowledge is obtained from the pathophysiological understanding of myocardial ischemia. The knowledge is then expressed mathematically. Typical physiological models of cardiovascular deterioration rely upon cardiovascular parameters, but typically not on respiratory or gas exchange parameters. Accordingly, the illustrative physiological model-based detector 162 receives as input the cardiovascular parameters 42 but not the at least one respiratory parameter 44 and not the at least one gas exchange parameter 46. (However, it is also contemplated for the physiological model-based detector to employ rules additionally operating on respiratory and/or gas exchange parameter(s)).

The outputs of the three detectors 52, 160, 162 are updated at each instance that a patient data record is presented/updated. Each detector 52, 160, 162 outputs an assessment (i.e. score) estimating the onset of ischemia. The three outputs are then aggregated via the scores combiner 164 to generate the ischemia score value 166. In some embodiments, the scores combiner 164 normalizes the input and output to produce the ischemia score 166 in the range [0%,100%] where a score of 0% indicates lowest estimated likelihood/severity of cardiac ischemia, while 100% represents highest likelihood/severity of cardiac ischemia. The ischemia score 166 may, in general, evolve over time as parameters such as heart rate, respiration rate, tidal volume, blood pressure, and so forth are updated by readings of the sensors 20, 22, 24 and/or as other inputs such as blood gas analysis results are input to the system.

The weights for the scores output by the respective detectors 52, 160, 162 are suitably determined (or fine-tuned) during the training phase by optimizing the fit between the output 166 and the annotated labels yᵢ pertaining to cardiac ischemia. The scores combiner 164 may employ a simple weighted average or weighted sum of the outputs of the constituent ischemia detectors 52, 160, 162. In other embodiments, the scores combiner 164 performs the weighted aggregation using a more sophisticated technique such as Linear Discriminator Analysis (LDA) to provide the single value 166 of ischemia detection.

As already mentioned, the output 166 may be employed in the context of the cEWS system 40 of FIG. 1, substituting for the output of the ischemia classifier 52 in this cEWS system 40. In other embodiments, the system of FIG. 4 operates as a stand-alone myocardial ischemia detector, and the level of detected ischemia severity can be displayed as a binary value (e.g. indicating possible cardiac ischemia if the aggregate score 166 is above a certain threshold), or quantized to one of more than two levels (multi-level discretized output) for example represented as a “traffic light” with green color indicating low ischemia likelihood/severity, yellow indicating moderate ischemia likelihood/severity, and red indicating high ischemia likelihood/severity. Additionally or alternatively, the ischemia score 166 may be presented as a numeric value (updated in real time), or as a trend line.

While the aggregate score 166 is expected to be more accurate than the individual outputs of the respective ischemia detector components 52, 160, 162, it is also contemplated to display the outputs of the individual ischemia detector components 52, 160, 162, for example using one of the above-mentioned binary, color coded, numeric, and/or trend line representations.

While illustrative FIG. 4 combines the empirical ischemia classifier 52, rules-based ischemia detector 160, and physiological model-based detector 162, it is alternatively contemplated to include only two of these components 52, 160, 162. For example, the physiological model-based detector 162, is optionally omitted. Furthermore, as already mentioned, it will be appreciated any of the other illustrative cardiovascular deterioration modality classifiers 54, 56, 58 may be similarly modified to further incorporate a rule-based detector component and/or a physiological model-based detector component.

It will be appreciated that the disclosed cardiovascular deterioration early warning system cEWS system 40, and/or the constituent classifiers 52, 54, 56, 58, 152 or stand-alone modality detector 152, may also be embodied as a non-transitory storage medium storing instructions readable and executable by the microprocessor or microcontroller of the patient monitor 12, or by another electronic data processing device, to perform the disclosed cardiovascular deterioration detection operations. Such a non-transitory storage medium may, by way of illustration, include: a hard disk drive or other magnetic storage medium; an optical disk or other optical storage medium; a read-only memory (ROM), electronically programmable read-only-memory (PROM), flash memory or other electronic storage medium; various combinations thereof; and so forth.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A patient monitor comprising:
a display component;
a plurality of sensors reading vital signs of a human subject including at one cardiovascular parameter and at least one respiratory parameter; and
a microprocessor or microcontroller programmed to perform a cardiovascular early warning scoring (cEWS) method including the operations of:
(i) classifying the human subject using a plurality of cardiovascular deterioration classifiers each trained to classify the human subject respective to a different type of cardiovascular deterioration to generate cardiovascular early warning scores for the different types of cardiovascular deterioration, the plurality of cardiovascular deterioration classifiers operating on a set of inputs characterizing the human subject including the at least one cardiovascular parameter and the at least one respiratory parameter read by the plurality of sensors, and
(ii) outputting the cardiovascular early warning scores for the different types of cardiovascular deterioration on the display component of the patient monitor.

2. The patient monitor of claim 1 wherein the plurality of cardiovascular deterioration classifiers operate on the set of inputs including the at least one cardiovascular parameter read by electrocardiograph electrodes and the at least one respiratory parameter comprising tidal volume read by an airflow sensor.

3. The patient monitor of claim 2 wherein the at least one respiratory parameter further includes respiration rate.

4. The patient monitor of claim 1 wherein the set of inputs characterizing the human subject further include at least one gas exchange parameter read by the plurality of sensors.

5. The patient monitor of claim 4 wherein the plurality of cardiovascular deterioration classifiers operate on the set of inputs including the at least gas exchange parameter comprising saturation of peripheral oxygen (SpO₂) read by a pulse oximeter sensor.

6. The patient monitor of claim 1 wherein the plurality of cardiovascular deterioration classifiers operate on the set of inputs characterizing the human subject further including blood gas analysis test results including at least one of partial pressure of oxygen (PaO₂) and partial pressure of carbon dioxide (PaCO₂),
wherein the blood gas analysis test results are input to the patient monitor by one of a user input device and reading an Electronic Health Record or Electronic Medical Record via an electronic data network.

7. The patient monitor of claim 6 wherein the plurality of cardiovascular deterioration classifiers operate on the set of inputs including said blood gas analysis test results further including troponin level in the blood.

8. The patient monitor of claim 1 wherein the plurality of cardiovascular deterioration classifiers include at least two classifiers of the group of cardiovascular deterioration classifiers consisting of a myocardial ischemia, a left ventricular hypertrophy classifier, a systolic heart failure classifier, and a diastolic heart failure classifier.

9. The patient monitor of claim 1 wherein the plurality of cardiovascular deterioration classifiers includes a first cardiovascular deterioration classifier classifying the human subject respective to a first type of cardiovascular deterioration to generate a cardiovascular early warning score for the first type of cardiovascular deterioration, wherein the first cardiovascular deterioration classifier comprises:
an empirical classifier trained using labeled training data to generate an empirical score for the first type of cardiovascular deterioration;
a rules-based cardiovascular deterioration detector applying a set of rules to generate a rules-based score for the first type of cardiovascular deterioration; and
a scores combiner generating a weighted combination of scores for the first type of cardiovascular deterioration including at least the empirical score and the rules-based score.

10. (canceled)

11. The patient monitor of claim 9 wherein the first cardiovascular deterioration classifier further comprises:
a physiological model-based detector modeling the first type of cardiovascular deterioration using algebraic or differential equations to generate a model-based score for the first type of cardiovascular deterioration;
wherein the scores combiner generates the weighted combination of scores for the first type of cardiovascular deterioration including the empirical score, the rules-based score, and the model-based score.

12. A non-transitory storage medium storing instructions readable and executable by a patient monitor comprising a plurality of sensors, a display component, and a microprocessor or microcontroller to perform a myocardial ischemia early warning method including the operations of:
acquiring vital sign data for a human subject using the plurality of sensors;
classifying the human subject to generate an empirical myocardial ischemia score using an empirical myocardial ischemia classifier trained on a labeled data set representing training subjects with each training subject i represented by a vector x̅ᵢ of features of the training subject i and a label yᵢ representing a state of myocardial ischemia in the training subject i, the classifying including inputting a vector to the empirical myocardial ischemia classifier that includes features generated from the acquired vital sign data for the human subject;
generating at least one additional myocardial ischemia score by applying a set of rules or a physiological model to a set of inputs characterizing the human subject including inputs generated from the acquired vital sign data for the human subject;
generating a combined myocardial ischemia score comprising a weighted combination of the empirical myocardial ischemia score and the at least one additional myocardial ischemia score; and
displaying a representation of the combined myocardial ischemia score on the display component of the patient monitor.

13. The non-transitory storage medium of claim 12 wherein the operation of generating at least one additional myocardial ischemia score includes:
generating a rules-based myocardial ischemia score by applying a set of rules to the set of inputs characterizing the human subject.

14. (canceled)

15. (canceled)

16. The non-transitory storage medium of claim 12 wherein the operation of generating at least one additional myocardial ischemia score includes:
generating a physiological model-based myocardial ischemia score using a physiological model of myocardial ischemia operating on the set of inputs characterizing the human subject.

17. The non-transitory storage medium of claim 12 wherein the operation of generating a combined myocardial ischemia score includes:
generating the combined myocardial ischemia score combining the empirical myocardial ischemia score and the at least one additional myocardial ischemia score using Linear Discriminator Analysis.

18. The non-transitory storage medium of claim 12 wherein the operation of displaying a representation of the combined myocardial ischemia score on the display component of the patient monitor includes:
discretizing the combined myocardial ischemia score to generate a discretized combined myocardial ischemia score; and
displaying a representation of the discretized combined myocardial ischemia score on the display component of the patient monitor.

19. (canceled)

20. (canceled)
